SEQUENCE LISTING (2 sequences)

SEQ ID NO: 1 - protein, 6 aa, Artificial Sequence
  Short activating peptide
SLIGKV

SEQ ID NO: 2 - protein, 13 aa, Artificial Sequence
  Synthetic pan DR epitope
AKXVAAWTLKAAA

(19) United States
(12) Patent Application Publication
(10) Pub. No.: US 2010/0015160 A1
(43) Pub. Date: Jan. 21, 2010

(54) COMPOSITIONS AND METHODS FOR DIAGNOSING AND TREATING ENDOMETRIOSIS

(75) Inventors: Charles Lockwood, Madison, CT (US); Graciela Krikun, Hamden, CT (US)

Correspondence Address: Pabst Patent Group LLP, 1545 PEACHTREE STREET NE, SUITE 320, ATLANTA, GA, 30309, US

(73) Assignee: Yale University

(21) Appl. No.: 12/527,609

(22) PCT Filed: Feb. 21, 2008
(86) PCT No.: PCT/US08/54559

§ 371 Date: Aug. 18, 2009

Related U.S. Application Data
(60) Provisional application No. 60/902,570, filed on Feb. 21, 2007.

(51) Int. Cl. A61K 39/395; A61K 31/7088; C12Q 1/68; A61P 43/00

(52) U.S. Cl. 424/141.1; 514/44 R; 514/44 A; 435/6

(57) ABSTRACT

Although TF is expressed on perivascular cells of normal tissues and in the adventitial layer of blood vessels, these cells are sequestered from contact with circulating fVII by the tight endothelial cell layer of the normal vasculature. Therefore, differential expression of TF by endometriotic tissue makes it a specific target for inhibiting or treating endometriosis. Similarly, overexpression of PAR-2 by endometrial tissue in women with endometriosis makes it a target for inhibiting or treating endometriosis. In one embodiment, interference with binding of fVII to TF or of the TF/fVIIa to PAR-2 is accomplished by providing one or more antagonists that reduce or inhibit binding of these proteins as described above. In another embodiment, the catalytic activity of PAR-2 or the TF/fVIIa complex is inhibited by providing one or more antagonists as disclosed above. In another embodiment, TF and/or PAR-2 expression is downregulated by providing one or more inhibitory nucleic acids including, but not limited to, ribozymes, triplex-forming oligonucleotides (TFOs), antisense DNA, external guide sequences (EGSs)f siRNA, and microRNA specific for nucleic acids encoding TF or PAR-2. TF and PAR-2 antagonists can also be provided in combination with other anti-angiogenic agents or other agents used to treat endometriosis, such as those described above.

### CROSS-REFERENCE TO RELATED APPLICATIONS

This application claims priority to and benefit of U.S. Provisional Patent Application No. 60/902,570 filed on Feb. 21, 2007.

### FIELD OF THE INVENTION

Aspects of this invention are generally related to compositions and methods for the inhibition, treatment and diagnosis of endometriosis.

### BACKGROUND OF THE INVENTION

Endometriosis is a gynecological disorder characterized by the presence of endometrial tissue in extra-uterine sites. Endometrial lesions are primarily located on the pelvic peritoneum and the ovaries, but can also be found in the pericardium, pleura, lung parenchyma and, rarely, the brain. Endometriosis is an estrogen driven disease and therefore affects almost exclusively women during their reproductive years. Endometrial implants can result in substantial morbidity, including pelvic adhesions and pain, painful menstrual periods, fatigue, bowel problems and infertility are typical and often devastating symptoms (Berkley et al., Science, 308(5728):1587-9 (2005)). The prevalence of endometriosis in reproductive-aged women is estimated at approximately 10%. However, in women undergoing laparoscopic surgery to investigate the cause of their infertility or pelvic pain, the rates are about 30% and 50% respectively Kennedy et al., Hum. Reprod., 20(10):2698-704 (2005)).

Identification of endometriotic lesions by laparoscopy and, if possible, histological verification of endometrial glands and stromal cells are required to make the diagnosis. The main pathological processes associated with the disease are peritoneal inflammation and fibrosis, and the formation of adhesions and ovarian cysts. In addition, endometriosis has been associated with an increased risk of developing clear cell and endometrioid ovarian carcinoma (Prowse et al., Int. J. Cancer, 119(3):556-62 (2006)). Complications associated with endometriosis often require extensive and oftentimes ineffective medical and surgical treatments. Hence, this disease is costly and both physically and psychologically debilitating.

Different hypotheses on the pathogenesis of endometriosis exist, which may be supplementary to each other (Dunselman and Groothuis, Gynecol. Obstet. Invest., 57(1):42-3 (2004)). First it is assumed that local or exogenous stimuli lead to metaplasia of the mesothelial cell layer of the peritoneal wall (Meyer, Geburtshilfe Gynakol., 49:32-41 (1903)). These metaplastic lesions are believed to grow by acquiring blood supply after local invasion of the basement membrane. Secondly, it has been suggested that endometriotic cells may be disseminated systemically through the lymphatic or blood vessel systems (Halban, Wien. Kin. Wochenschr., 37:1205-6 (1924)). A is theory could explain the occurrence of endometriotic lesions at distant sites such as the lungs, skin, brain and eye.

The other, more widely favored hypothesis, encompasses a phenomenon called retrograde menstruation (Sampson, Am. J. Obstet. Gynecol., 14:422-69 (1927)). It is believed that viable endometrial cells and tissue reach the abdomen through the Fallopian tubes at the time of menstruation, where they adhere to the peritoneal wall, invade the mesothelial cell layer and basement membrane through enzymatic degradation and provoke angiogenesis. However, invasion has only been shown in animal models, but not in humans (Dunselman and Groothuis, Gynecol. Obstet. Invest., 57(1):42-3 (2004)), Also, retrograde menstruation is a common phenomenon occurring in more than 90% of women (Halme et al., Obstet. Gynecol., 64(2):151-4 (1984)). Therefore, further conditions must be in place to support endometriotic growth.

Angiogenesis is the formation of new blood vessels from pre-existing vasculature, and is a process fundamental to the human menstrual cycle. The uterine endometrium is a dynamic tissue that undergoes regular cycles of growth and breakdown, and has long been recognized as one of the few adult tissues where significant angiogenesis occurs on a routine, physiological basis. While the physiological and molecular mechanisms by which endometriotic lesions are established are not entirely clear, it is now well recognized that angiogenesis plays a key role in the establishment and growth of endometriotic lesions, a concept widely accepted in tumor growth (Folkman, N. Engl. J. Med, 285(21):1182-6 (1971); Taylor, et al., Ann. N.Y. Acad. Sci., 955:89-100 (2002)). Endometriotic implants require neovascularization to survive, grow and invade ectopic sites, and there is general agreement that endometriosis is associated with a local inflammatory response and that vascularization at the site of invasion plays a decisive role in the pathogenesis of the disease. Peritoneal fluid from women with endometriosis is highly angiogenic. The molecular elements and mechanisms regulating angiogenesis that functions in the establishment, growth and persistence of endometriotic lesions are incompletely understood. Existing methods for treating or inhibiting endometriosis are largely ineffective and the molecular mechanisms underlying the disorder are not completely known. In addition, there is a need for molecular markers that can be used to definitively diagnose occurrences of endometriosis. It would therefore be advantageous to provide compositions and methods for the treatment and inhibition of endometriosis. It would also be advantageous to provide compositions and methods for the diagnosis of endometriosis.

Therefore, it is an object of the invention to provide compositions and methods of use thereof that inhibit or treat one or more symptoms associated with endometriosis.

It is another object of the invention to provide compositions and methods for inhibiting angiogenesis that supports the formation of endometriotic lesions.

It is yet another object of the invention to provide compositions and methods for targeting endometriotic endothelial cells for cytolysis by cells and molecules of the immune system.

It is still another object of the invention to provide compositions and methods for the detection or diagnosis of endometriosis.

It is still another object of the invention to provide methods for screening for compounds that inhibit or alleviate one or more symptoms associated with endometriosis.

### SUMMARY OF THE INVENTION

Although tissue factor (TF) is expressed on perivascular cells of normal tissues and in the adventitial layer of blood vessels, these cells are sequestered from contact with circulating fVII by the tight endothelial cell layer of the normal vasculature. Therefore, differential expression of TF by endometriotic endothelium make it an intravascular target while TF over-expressed on endometriotic macrophages, epithelial cells and stromal cells provides an lintraperitoneal target for inhibiting or treating endometriosis. Similarly, overexpression of PAR-2 by endometrial tissue in women with endometriosis makes it a target for inhibiting or treating endometriosis.

In one embodiment, interference with binding of fVII to TF or of the TF/fVIIa to PAR-2 is accomplished by providing one or more antagonists that reduce or inhibit binding of these proteins as described above. In another embodiment, the catalytic activity of PAR-2 or the TF/fVIIa complex is inhibited by providing one or more antagonists as disclosed above. In another embodiment, TF and/or PAR-2 expression is downregulated by providing one or more inhibitory nucleic acids including, but not limited to, ribozymes, triplex-forming oligonucleotides (TFOs), antisense DNA, external guide sequences EGSs), siRNA, and microRNA specific for nucleic acids encoding TF or PAR-2. TF and PAR-2 antagonists can also be provided in combination with other anti-angiogenic agents or other agents used to treat endometriosis, such as those described above.

The discovery that TF and PAR-2 are overexpressed in endometrial tissues from women with endometriosis also provides new markers for diagnosing endometriosis and/or determining the clinical stage of endometriosis in a subject, or progression of treatment.

### DETAILED DESCRIPTION OF THE INVENTION

### I. Definitions

Terms defined herein have meanings as commonly understood by a person of ordinary skill in the art.

As used herein, the terms “tissue factor antagonist”, “TF antagonist” or “PAR-2 antagonist” refers to compounds that inhibit, reduce, or block the biological activity or expression of tissue factor and/or PAR-2. Suitable TF and PAR-2 antagonists include, but are not limited to, antibodies and antibody fragments that bind tissue factor, fVII or PAR-2, other polypeptides that bind to tissue factor, fVII or PAR-2 and inhibit their activity, including factor VII variants, inhibitors of the catalytic activity of TF/fVIIa, small organic compounds, and inhibitory nucleic acids specific for tissue factor or PAR-2-encoding nucleic acids.

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the term “polypeptide” refers to a chain of amino acids of any length, regardless of modification (e.g., phosphorylation or glycosylation).

As used herein, a “variant” polypeptide contains at least one amino acid sequence alteration (addition, deletion, substitution, preferably conservative i.e., not substantially changing the function except in magnitude) as compared to the amino acid sequence of the corresponding wild-type polypeptide.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, “isolated nucleic acid” refers to a nucleic acid that is separated from other nucleic acid molecules that are present in a mammalian genome, including nucleic acids that normally flank one or both sides of the nucleic acid in a mammalian genome.

As used herein with respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “host cell” refers to prokaryotic and eukaryotic cells into which a recombinant expression vector can be introduced.

As used herein, “transformed” and “transfected” encompass the introduction of a nucleic acid (e.g. a vector) into a cell by a number of techniques known in the art.

The terms “individual”, “host”, “subject”, and “patient” are used interchangeably herein.

As used herein the term “effective amount” or “therapeutically effective amount” means a dosage sufficient to treat, inhibit, or alleviate one or more symptoms of endometriosis or to otherwise provide a desired pharmacologic and/or physiologic effect. The precise dosage will vary according to a variety of factors such as subject-dependent variables (e.g., age, immune system health, etc.), the disease, and the treatment being effected. The term “effective amount” is also used to refer to an amount of a composition sufficient to detect the presence of a tissue factor or PAR-2 polypeptide or nucleic acid molecule encoding a tissue factor or PAR-2 polypeptide in a specimen.

As used herein, the phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹, or 10¹⁰ M⁻¹. Affinities greater than 10⁸ M⁻¹ are preferred.

As used herein, the terms “antibody” or “immunoglobulin” are used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites, Bispecific antibodies can be produced by a variety of methods including fusion of hybridomas or linking of Fab′ fragments. See, e.g., Songsivilai and Lachmann, Clin. Exp. Immunol., 79:315-321 (1990); Kostelny et al., J. Immunol., 148:1547-1553 (1992).

As used herein, an “antigen” is an entity to which an antibody specifically binds.

As used herein, the terms “epitope” or “antigenic determinant” refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids, in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn B. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke, et al., J. Inf. Dis., 170:1110-19 (1994)), by antigen-dependent killing (cytotoxic T lymphocyte assay, Tigges, et al., J. Immunol., 156:3901-3910) or by cytokine secretion.

### Compositions for the Treatment of Endometriosis

Although it is now widely recognized that angiogenesis plays a critical role in the establishment, growth and persistence of endometriotic lesions, the molecular elements and mechanisms regulating angiogenesis in endometriosis are incompletely understood.

It has been discovered that tissue factor (TF) is overexpressed in epithelial cells, stromal cells, infiltrating macrophages, and endothelial cells in ectopic endothelium in women with endometriosis. Tissue factor is a cell membrane-bound glycoprotein (MW 46 kDa) comprised of a hydrophilic extracellular domain, a membrane-spanning hydrophobic domain, and a cytoplasmic tail of 21 residues, including a non-disulfide-linked cysteine (Bach, et al. J. Biol. Chem., 256(16):8324-31 (1981); Nemerson, Blood, 71(1):1-8 (1988); Nemerson and Pitlick, Prog. Hemost. Thromb., 1:1-37 (1972)).

Biological activity of the mature protein requires posttranslational modification to include carbohydrate moieties. Endothelial cells and other cells in contact with the circulation do not normally express TF. However, following vascular disruption, perivascular cell-bound TF binds to circulating factor Vlla to mediate the activation of both factor IX and X and ultimately to generate thrombin (Carson and Konigsberg, Thromb. Haemost., 44(1):12-5 (1980); Guha, et al., Proc. Natl. Acad. Sci. U.S.A., 83(2):299-302 (1986); Radcliffe and Nemerson, J. Biol. Chem., 250(2):388-95 (1975); Masys, et al., Blood, 60(5):1143-50 (1982)).

TF expression is limited to stromal cells of the secretory phase with far lower expression in glandular epithelium. Specifically, it has been shown that progesterone (P4) enhances endometrial stromal cell TF mRNA and protein expression in vitro and that immunohistochemical staining for TF protein and in situ hybridization signaling for TF mRNA were greatest in stromal cells from the P4-dominated secretory phase (Lockwood, et al., J. Clin. Endocrinol. Metab., 76(1):231-6 (1993); Lockwood, et al., J. Clin. Endocrinol. Metab., 77(4):1014-9 (1993); Lockwood, et al., Ann. N. Y. Acad. Sci., 828:188-93 (1997); Krikun, et al., J. Clin. Endocrinol Metab., 83(3):926-30 (1998); Lockwood, et al., J. Clin. Endocrinol. Metab., 85(1):297-301 (2000); Krikun, et al., Mol. Endocrinol., 14(3):393-400 (2000); Runic, et al., J. Clin. Endocrinol. Metab., 82(6):1983-8 (1997). In contrast, the examples below indicate that in endometriosis, TF is greatly over-expressed in both glandular epithelium and stromal cells irrespective of menstrual phase. Tissue factor immunostaining was also observed in macrophages infiltrating endometriotic tissues.

In addition, to its role in hemostasis, TF/VIIa binding has important coagulation-independent functions, especially in embryonic and oncogenic angiogenesis, leukocyte diapedesis and inflammation (Versteeg, et al., Carcinogenesis, 24(6):1009-13 (2003)). Indeed, TF deficiency causes embryonic lethality in the mouse. Tissue factor null (TF^{-/-}) embryos die at embryonic day E10.5 and display disorganization of the yolk sac vasculature suggesting that TF plays a pivotal role in vasculogenesis (Carmeliet, et al., Nature, 383(6595):73-5 (1996); Toomey, et al., Blood, 88(5):1583-7 (1996)). The absence of reports of TF deficiency in humans suggests a parallel obligatory requirement.

While TF/VIIa signaling plays a critical role in angiogenesis, the underlying molecular mechanisms are not completely understood. One mechanism by which the TF/VIIa complex is thought to mediate angiogenesis is through the proteinase activated receptor-2 (PAR-2) (Ahamed and Ruf, J. Biol. Chem., 279(22):23038-44 (2004); Hjortoe, et al., Blood, 103(8):3029-37 (2004); Nystedt, et al., J. Biol. Chem., 271(25):14910-5 (1996)).

In addition to overexpression of TF in endometriotic tissues, it has also been discovered that PAR-2 is overexpressed in glandular epithelial and endothelial cells of eutopic and ectopic endothelium in women with endometriosis.

PAR-2 is a seven transmembrane G-protein coupled receptor (GPCR) which signals in response to the proteolytic activity of trypsin, tryptase, matriptase, the TF/fVIIa complex and other proteases such as neutrophil protease-3. Proteolytic cleavage of the amino terminus results in the unveiling of a new amino terminus that activates the receptor through a tethered peptide ligand mechanism; essentially the terminus becomes the ligand which inserts into the ligand binding pocket of the receptor. The short activating peptide SLIGKV (SEQ ID NO:1) produced by Neosystem SA, France, activates the human PAR-2 receptor. Upon binding of the ligand, there is an increase in intracellular calcium concentration.

Several studies have demonstrated that PAR-2 is involved in angiogenesis, neovascularization and inflammation. PAR-2 has also been associated with pain transmission, tissue injury and regulation of cardiovascular function. For example, Milia, et al., Circulation Research, 91(4):346-352 (2002), discuss the wide expression of PAR-2 in the cardiovascular system, mediation of endothelial cell mitogenesis in vitro by PAR-2, and promotion of vasodilation and microvascular permeability in vivo by PAR-2: all of these steps are regarded as essential steps in angiogenesis.

In addition to signaling through PAR-2, the TF/fVIIa complex also indirectly stimulates angiogenesis through the generation of thrombin. Compositions including one or more TF or PAR-2 antagonists are provided herein. Tissue factor and PAR-2 antagonists include compounds that inhibit, reduce, or block the biological activity or expression of TF and/or PAR-2.

In certain embodiments, the compositions include as an active agent one or more TF and/or PAR-2 antagonists in an effective amount to inhibit, reduce, alleviate, or decrease one or more symptoms associated with endometriosis.

Compositions that Reduce or Inhibit the Function of Tissue Factor or PAR-2

Tissue factor and PAR-2 antagonists that reduce or inhibit a biological

function of TF or PAR-2 can be competitive or noncompetitive inhibitors. Tissue factor and PAR-2 antagonists preferably inhibit a biological activity of TF or PAR-2 by at least 20%, more preferably by at least 30%, more preferably by at least 40%, 50%, 60%, 70%, 80%, 90%, 95%, 86%, 97%, 98%, 99%, or more.

In some embodiments, TF and PAR-2 antagonists are capable of reducing or inhibiting one or more activities stimulated by TF or PAR-2 in cells expressing these molecules on their surface. In some embodiments, the cell is an epithelial cell, an endothelial cell, macrophage, or a stromal cell of ectopic endometrium. In a preferred embodiment, the cells are present in endometriotic lesions in women with endometriosis.

In another embodiment, TF and PAR-2 antagonists are capable of reducing or inhibiting one or more catalytic activities of TF or PAR-2. In a preferred embodiment, the catalytic activity that is inhibited is proteolysis. In other embodiments, TF and PAR-2 antagonists are capable of reducing or inhibiting the binding of TF to fVII or the binding of the TF/fVIIa complex to PAR-2.

### Antibodies

In one embodiment, TF and PAR-2 antagonists are antibodies. These antibodies are referred to herein as “blocking”, “function-blocking” or “antagonistic” antibodies. Antibodies or antibody fragments that specifically bind to TF, fVII or PAR-2 can be used to reduce or inhibit the binding of the TF to fVII or of the TF/VIIa complex to PAR-2. Antibodies or antibody fragments that specifically bind to TF, fVII or PAR-2 can also be used to reduce or inhibit the catalytic activity of the TF/VIIa complex or PAR-2. Methods of producing antibodies are well known and within the ability of one of ordinary skill in the art and are described in more detail below. Many suitable antagonistic antibodies that bind to TF and PAR-2 are known in the art.

In preferred embodiments the antagonistic antibodies specifically bind to a portion of the extracellular domain of TF or to an extracellular domain of PAR-2.

The immunogen used to generate the antibody may be any immunogenic portion of TF, fVII or PAR-2. Preferred immunogens include all or a part of the extracellular domain of human TF or extracellular domains of PAR-2, where these residues contain the post-translational modifications, such as glycosylation, found on native TF or PAR-2. Immunogens including the extracellular domain or immunogenic fragments thereof are produced in a variety of ways known in the art, e.g., expression of cloned genes using conventional recombinant methods, synthesized peptide complexes, isolation from cells of origin, cell populations expressing high levels of TF or PAR-2.

The antibodies disclosed herein are capable of binding to a polypeptide having at least about 70%, more preferably 75%, 80%, 85%, 90%, 95% identity to human TF, as found at GENBANK accession number MIM: 134390

GeneID: 2152, NT_{—}032977 or PAR-2, as found at GENBANK accession number MIM: 600933 GeneID: 2150.

The antibodies may be polyclonal or monoclonal antibodies. The antibodies may be xenogeneic, allogeneic, syngeneic, or modified forms thereof, such as humanized, single chain, antibody fragments or chimeric antibodies. The antibodies may also be antiidiotypic antibodies. Antibodies, as used herein, also includes antibody fragments including Fab and F(ab)₂ fragments, and antibodies produced as a single chain antibody or scFv instead of the normal multimeric structure. The antibodies may be an IgG such as IgG1, IgG2, IgG3 or IgG4; or IgM, IgA, IgE or IgD isotype. The constant domain of the antibody heavy chain may be selected depending on the effector function desired. The light chain constant domain may be a kappa or lambda constant domain.

### Other Polypeptides

In another embodiment, TF and PAR-2 antagonists are polypeptides other than antibodies that bind to TF or PAR-2. Tissue factor- or PAR-2-binding polypeptides can be used to reduce or inhibit the binding of TF to fVII or of the TF/fVIIa complex to PAR-2. Tissue factor and PAR-2 antagonists can also be used to inhibit catalytic activities of TF/fVIIa or PAR-2. Methods of producing polypeptides are well known and within the ability of one of ordinary skill in the art and are described in more detail below.

In some embodiments the polypeptides are soluble fragments of full length TF, fVII or PAR-2 polypeptides or full length fVII polypeptides. As used herein, a fragment of TF, fVII or PAR-2 refers to any subset of the polypeptide that is less amino acids than the full length protein. Soluble fragments generally lack some or all of the intracellular and/or transmembrane domains. In some embodiments, soluble fragments of TF, fVII or PARE-2 include all or a fragment of the extracellular domain of TF or fragments of extracellular domains of PAR-2.

In one embodiment the TF antagonist is a catalytically inactive fVIIa polypeptide. As used herein, the terms “factor VII” or “fVII” refer to fVII polypeptides in their uncleaved (zymogen) form. The terms “factor VIIa” or “fVIIa” refer to native bioactive forms of fVII. Typically, fVII is cleaved between residues 152 and 153 to yield fVIIa. When referring to inactive factor VII polypeptides that can be used as TF antagonist, the terms “fVII” and “fVIIa” are used interchangeably except if noted otherwise.

Inactive fVIIa polypeptides can be one or more chemically inactivated fVII molecules in which the active site is covalently modified by interaction with one or more covalent active site inhibitors. Alternatively, inactive fVIIa polypeptides can be generated by one or more amino acid substitutions, insertions or deletions that alter the catalytic activity of the active site of the polypeptide. The term “active site” when used herein with reference to fVIIa refers to the catalytic and zymogen substrate binding site, including the “S₁” site of fVIIa (Hu and Garen, Proc Natl Acad Sci U S A., 98:12180-5 (2001). Inactive fVIIa polypeptides can have very high affinity for TF as compared to the binding of native fVII. Inactive fVIIa polypeptides can therefore effectively compete with native fVII for binding to TF. Additionally, catalytically inactive fVII polypeptides reduce activation of the coagulation pathway which can be deleterious. The catalytic activity of fVII polypeptides can be tested by measuring the fVIIa-catalyzed conversion of fX to fXa using standard assays.

Chemical moieties used to inactivate fVII molecules include irreversible fVIIa serine protease inhibitors. Such irreversible active site inhibitors generally form covalent bonds with the protease active site. Such irreversible inhibitors include, but are not limited to, general serine protease inhibitors such as peptide chloromethylketones (Williams, et al., J. Biol. Chem., 264:7536-7540 (1989)) or peptidyl chloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate DFP); tosylpropylchloromethyl ketone (TPCK); tosyllysylchloromethyl ketone (TLCK); nitrophenylsulphonates and related compounds; heterocyclic protease inhibitors such as isocoumarines, and coumarins.

Examples of peptidic irreversible fVIIa inhibitors include, but are not limited to, Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone, and Dansyl-D-Glu-Gly-Arg chloromethylketone.

Other exemplary fVIIa inhibitors also include benzoxazinones or heterocyclic analogues thereof such as described in PCT/DK91/00138. An agent which displays great potency toward VIIa inhibition is G17905 TF·FVIIa (Ki=0.35±0.11 nM), G17905 effectively inhibited thrombus formation in a baboon arterio-venous shunt model, reducing platelet and fibrin deposition by 70% at 0.4 mg/kg+0.1 mg/kg/min infusion. (Olivero et al, J. Biol. Chem., 280, 9160-9169).

Examples of other fVIIa inhibitors include, but are not limited to, small peptides including, but not limited to, Phe-Phe-Arg, D-Phe-Phe-Arg, Phe-Phe-Arg, D-Phe-Phe-Arg, Phe-Pro-Arg, D-Phe-Pro-Arg, Phe-Pro-Arg, D-Phe-Pro-Arg, L- and D-Glu-Gly-Arg; peptidomimetics; benzamidine systems; heterocyclic structures substituted with one or more amidino groups; and substituted aromatic or heteroaromatic systems.

In another embodiment the TF antagonist is a variant fVII polypeptide that contains one or more amino acid sequence alterations relative to native fVII and/or contains truncated amino acid sequences relative to native fVII (i.e., fVII fragments). In a preferred embodiment, fVIIa variants or fragments have reduced proteolytic activity when compared with native fVIIa.

As used herein, a “variant” polypeptide contains at least one amino acid sequence alteration as compared to the amino acid sequence of the corresponding wild-type polypeptide. An amino acid sequence alteration can be, for example, a substitution, a deletion, or an insertion of one or more amino acids.

Antagonistic polypeptides can have any combination of amino acid substitutions, deletions or insertions. In one embodiment, isolated fVII antagonistic variant polypeptides have an integer number of amino acid alterations such that their amino acid sequence shares at least 60, 70, 80, 85, 90, 95, 97, 98, 99, 99.5 or 100% identity with an amino acid sequence of a corresponding wild type amino acid sequence. In a preferred embodiment fVII antagonistic variant polypeptides have an amino acid sequence sharing at least 60, 70, 80, 85, 90, 95, 97, 98, 99, 99.5 or 100% identity with the amino acid sequence of a corresponding wild type polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)); 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity (the number of identical residues)/(alignment length in amino acid residues)*100. For this calculation, alignment length includes internal gaps but does not include terminal gaps.

Amino acid substitutions in antagonistic variant polypeptides may be “conservative” or “non-conservative”. As used herein “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain e.g., phenylalanine, is substituted for (or by) a residue that does not have a side chain, e.g., glycine.

In one embodiment, the TF antagonist is a human fVIIa-derived peptide, which includes an fVII amino acid sequence that has an amino acid substitution of the lysine corresponding to position 341 of native human fVII. In another embodiment, the TF antagonist is a human fVIIa-derived peptide, which includes an fVII amino acid sequence that has an amino acid substitution of the serine corresponding to position 344 of native human factor VII. In another aspect, the TF antagonist is a human fVIIa-derived peptide, which includes an fVII sequence that also or alternatively has an amino acid substitution of the aspartic acid corresponding to position 242 of native human factor VII. In yet another aspect, the TF antagonist is a human fVIIa-derived peptide, which includes an fVII amino acid sequence that also or alternatively has an amino acid substitution of the histidine corresponding to position 193 of native human factor VII. Such peptides can correspond to native Evil in length, correspond to active FVII fragments, or be fusion proteins comprising a full length or truncated FVII sequence modified as indicated. In one embodiment, the TF antagonist is fVII-(K341A), fVII-(S344A), fVII-(D242A), and/or fVII-(H193A) or is a fVIIa polypeptide that contains any combination of these amino acid substitutions.

Other suitable fVIIa/TF polypeptides which inhibit the catalytic activity of the fVIIa/TF complex include members of the family of nematode-extracted anti-coagulant proteins (“NAPs”.) The discovery of NAPs was prompted by the observation that haematophagous hookworm parasites (e.g. Ancylostoma caninum) produce potent substances that prevents the coagulation of blood, leading to the discovery of a family of small proteins, designated NAPs. One of these proteins, designated NAPc2, was identified in the hookworm A. caninum using molecular cloning and subsequently produced as an 85-amino acid recombinant protein (rNAPc2) using the yeast Pichia pastoris. Both natural and rNAPc2 have been shown to potently inactivate the catalytic complex of fVIIa/TF by a unique mechanism that requires the initial binding of rNAPc2 to FX or fXa prior to the formation of a final quaternary inhibitory complex with fVIIa/TF (fVIIa/TF-Xa-rNAPc2).

Other suitable polypeptides include tissue factor pathway inhibitor (“TFPI”) and TFPI-2. TFPIs are plasma Kunitz-type serine protease inhibitors which modulate the initiation of coagulation induced by TF. In a factor fXa-dependent feedback system, TFPI binds directly and inhibits the TF/fVIIa complex, Normally, TFPI exists in plasma both as a full-length molecule and as variably carboxy-terminal truncated forms.

NAPs and TFPI can be variant polypeptides containing conservative or non-conservative amino acid substitutions, as described above, or can be fragments of full length NAPs or TFPI that retain the ability to inhibit the TF/fVIIa complex.

Suitable polypeptides that can be used to inhibit or reduce PAR-2 activity include fragments, variants and derivatives of the agonist peptide, such as SLIGKV (SEQ ID NO: 1) obtained from Neosystem SA, France, that retain the ability to bind to the receptor, but do not cause significant activation of the receptor. Several fragments and derivatives of the SLIGKV (SEQ ID NO: 1) peptide that function as inhibitors of PAR-2 are disclosed in U.S. Published Application No. 2006/0142203. Additional peptides that function as inhibitors of PAR-2 are disclosed in U.S. Published Application No. 2006/0104944.

### Chemical Modifications of Polypeptides

Antagonistic polypeptides may also be modified by chemical moieties that may be present in polypeptides in a normal cellular environment, for example, phosphorylation, methylation, amidation, sulfation, acylation, glycosylation, sumoylation and ubiquitylation. Polypeptides may also be modified with a label capable of providing a detectable signal, either directly or indirectly, including, but not limited to, radioisotopes and fluorescent compounds.

Antagonistic polypeptides may also be modified by chemical moieties that are not normally added to polypeptides in a cellular environment. Such modifications may be introduced into the molecule by reacting targeted amino acid residues of the polypeptide with an organic derivatizing agent that is capable of reacting with selected side chains or terminal residues. Another modification is cyclization of the protein.

Examples of chemical derivatives of the polypeptides include lysinyl and amino terminal residues derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Carboxyl side groups, aspartyl or glutaryl, may be selectively modified by reaction with carbodiimides (R—N═C═N—R′) such as 1-cyclohexyl-3-(2-morpholinyl-(4-ethyl)carbodiimide or 1-ethyl-3-(4-azonia-4,4-dimethylpentyl) carbodiimide. Furthermore, aspartyl and glutamyl residues can be converted to asparaginyl and glutaminyl residues by reaction with ammonia Polypeptides may also include one or more D-amino acids that are substituted for one or more L-amino acids.

### Fusion Polypeptides

The polypeptides may be coupled to other polypeptides to form fusion proteins, for example, having a first fusion partner fused (i) directly to a second polypeptide or, (ii) optionally, fused to a linker peptide sequence that is fused to the second polypeptide. The presence of the second polypeptide fusion partner can alter the solubility, affinity and/or valency of the first fusion partner. As used herein, “valency” refers to the number of binding sites available per molecule. The first fusion partner can be any of the polypeptides disclosed above, including fragments and variants and chemical modification. In a preferred embodiment the first fusion partner includes all or a part of a variant fVII polypeptide. Variant fVII fusion proteins described herein include any combination of amino acid alterations (i.e. substitution, deletion or insertion), fragments of fVII, and/or chemical modifications as described above. In a particularly preferred embodiment, the variant fVII polypeptide contains a K341A amino acid substitution.

The second polypeptide binding partner may be N-terminal or C-terminal relative to the first fusion polypeptide. In a preferred embodiment, the second polypeptide is C-terminal to the first fusion polypeptide.

A large number of polypeptide sequences that are routinely used as fusion protein binding partners are well known in the art. Examples of useful polypeptide binding partners include, but are not limited to, green fluorescent protein (GFP), glutathione S-transferase (GST), polyhistidine, myc, hemagglutinin, Flag™ tag (Kodak, New Haven, Conn.), maltose E binding protein and protein A.

In a preferred embodiment, the first polypeptide fusion partner is fused to one or more domains of an immunoglobulin (Ig) heavy chain constant region (Fc effector domain), preferably having an amino acid sequence corresponding to the hinge, C_{H}2 and C_{H}3 regions of an immunoglobulin chain. The Fc effector domain provides cysteine residues that participate in disulfide bonds and cause the fusion polypeptides to dimerize, thereby increasing the valency of the first fusion partner. The Fc effector domain can be from any species, but preferably is derived from a human immunoglobulin.

### Small Molecules and Other Antagonists

A number of small molecule antagonists of TF are known in the art. For example, bezofuran, acylsulfamide and amidine inhibitors are described in U.S. Published Application Nos. 2007/0049601, 2007/0037814 and 2002/0055469, respectively. Additional bioactive agents may be screened for antagonistic activity against TF or PAR-2. In one embodiment, candidate bioactive agents are screened for their ability to reduce binding of fVII to TF or the TF/fVIIa complex to PAR-2. In another embodiment, candidate bioactive agents are screened for their ability to reduce proteolytic activity of either TF/fVIIa or PAR-2. The assays preferably utilize human proteins, although other proteins from other species may also be used.

The term “candidate bioactive agent” as used herein describes any molecule, e.g., protein, small organic molecule, carbohydrates (including polysaccharides), polynucleotide, lipids, etc. Generally a plurality of assay mixtures are run in parallel with different agent concentrations to obtain a differential response to the various concentrations. Typically, one of these concentrations serves as a negative control, i.e., at zero concentration or below the level of detection. In addition, positive controls, i.e. the use of agents known to bind fVII, TF or PAR-2 may be used.

Candidate agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides, e.g., peptidomimetics. Peptidomimetics can be made as described, e.g., in WO 98156401.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extras are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs. In a preferred embodiment the candidate bioactive agents are organic chemical moieties or small molecule chemical compositions, a wide variety of which are available in the art.

Compositions that Reduce or Bit the Expression of Tissue Factor or PAR-2

In another embodiment TF and PAR-2 antagonists reduce or inhibit the expression of TF or PAR-2. Antagonists that reduce or inhibit expression of TF or PAR-2 include inhibitory nucleic acids, including, but not limited to, ribozymes, triplex-forming oligonucleotides (TFOs), external guide sequences (EGSs) that promote cleavage by RNase P, peptide nucleic acids, antisense DNA, siRNA, and microRNA specific for nucleic acids encoding TF or PAR-2.

Useful inhibitory nucleic acids include those that reduce the expression of RNA encoding TF or PAR-2 by at least 20%, 30%, 40%, 50%, 60%, 70%, 80%, 90% or 95% compared to controls. Expression of TF or PAR-2 can be measured by methods well know to those of skill in the art, including northern blotting and quantitative polymerase chain reaction (PCR).

Inhibitory nucleic acids and methods of producing them are well known in the art. siRNA design software is available for example at http://i.cs.hku.hk/˜sirna/software/sirna.php. Synthesis of nucleic acids is well known see for example Molecular Cloning: A Laboratory Manual (Sambrook and Russel eds. 3^{rd} ed.) Cold Spring Harbor, N.Y. (2001). The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp, preferably 15 to 35 bp, and more preferably 21 to 30 bp long. Alternatively, the double-stranded RNA portion of a final transcription product of siRNA to be expressed can be, for example, 15 to 49 bp, preferably 15 to 35 bp, and more preferably 21 to 30 bp long. The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, the double-stranded RNA region of siRNAs in which two RNA strands pair up may contain both bulge and mismatched, which sum up to, preferably 1 to 7, more preferably 1 to 5 in number.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNA effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 bp double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the siRNA is not necessarily the cut off structure at both ends as described above, and may have a stem-loop structure in which ends of one side of double-stranded RNA are connected by a linker RNA. The length of the double-stranded RNA region (stem-loop portion) can be, for example, 15 to 49 bp, preferably 15 to 35 bp, and more preferably 21 to 30 bp long. Alternatively, the length of the double-stranded RNA region that is a final transcription product of siRNAs to be expressed is, for example, 15 to 49 bp, preferably 15 to 35 bp, and more preferably 21 to 30 bp long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA or viral RNA, or an artificial RNA molecule.

MiRNAs are produced by the cleavage of short stem-loop precursors by Dicer-like enzymes; whereas, siRNAs are produced by the cleavage of long double-stranded RNA molecules. MiRNAs are single-stranded, whereas siRNAs are double-stranded.

Methods for producing siRNA are known in the art. Because the nucleotide sequences that encode TF and PAR-2 are known, one of skill in the art could readily produce siRNAs that downregulate TF or PAR-2 expression in a host using the information that is publicly available.

### Additional Therapeutic Compounds

In certain embodiments, the TF and PAR-2 antagonists disclosed herein, including antagonistic TF and PAR-2 antibodies and fusion polypeptides, may be combined with one or more additional therapeutic agents.

### Other Anti-Angiogenic Compositions

The one or more additional therapeutic agents can include agents that modulate angiogenesis. Angiogenic inhibitors are known in the art and can be prepared by known methods. Many angiogenic inhibitors are available through commercial sources. For example, angiogenic inhibitors include integrin inhibitory compounds such as, αv integrin inhibitory antibodies, cell adhesion proteins, or functional fragments thereof which contain a cell adhesion binding sequence. Additional angiogenic inhibitors include, for example, angiostatin (see, e.g., U.S. Pat. No. 5,639,725), functional fragments of angiostatin, endostatin (see, e.g., WO 97/15666), fibroblast growth factor (FGF) inhibitors, FGF receptor inhibitors, VEGF inhibitors (VEGF antibodies, VEGF trap, VEGF receptor blockers, and other mechanisms of VEGF inhibition), thrombospondin, platelet factor 4, interferon, interleukin 12, thalidomide, some of the tetracyclines, and compounds involved in other mechanisms for the inhibition of angiogenesis. For a description of angiogenic inhibitors and targets set forth above, see, for example, Chen, et al., Cancer Res., 55:4230-4233 (1995), Good, et al., Proc. Natl. Acad. Sci. U.S.A., 87:6629-6628 (1990), O'Reilly, et al., Cell, 79:315-328 (1994), Parangi, et al., Proc. Natl. Acad. Sci. U.S.A., 93:2002-2007 (1996), Rastinejad, et al., Cell, 56:345-355 (1989), Gupta, et al., Proc. Nat. Acad. Sci. U.S.A., 92:7799-7803 (1995), Maione, et al., Science, 247:77-79 (1990), Angiolillo, et al., J. Exp. Med., 182:155-162 (1995), Strieter, et al., Biochem. Biophys. Res. Comm., 210:51-57 (1995); Voest, et al., J. Natl. Cancer Inst., 87:581-586 (1995), Cao, et al., J. Exp. Med, 182:2069-2077 (1995), Clapp, et al., Endocrinology, 133:1292-1299 (1993), Blood, et al., Bioch. Biophys Ata., 1032:89-118 (1990), and Moses, et al., Science 248:1408-1410 (1990).

### Other Compositions Used for Endometriosis Therapy

The one or more additional therapeutic agents can also or alternatively include agents that are routinely used for treatment or prevention of endometriosis. Suitable additional therapeutic agents include, but are not limited to, combined oral contraceptive pills, progestins, including dydrogesterone, medroxyprogesterone, norethisterone and levonorgestrel, GnRH agonists including buserelin, goserelin, leuprorelin, naferelin and triptorelin, synthetic androgens including danazol, and aromatase inhibitors.

Pharmaceutical Formulations

Pharmaceutical compositions including TF and PAR-2 antagonists, and vectors containing nucleic acids that encode TF and PAR-2 antagonists are provided. The pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration.

### i. Formulations for Parenteral Administration

In a preferred embodiment, the peptides are administered in an aqueous solution, by parenteral or peritoneal injection. The formulation may also be in the form of a suspension or emulsion. In general, pharmaceutical compositions are provided including effective amounts of a TF or PAR-2 antagonist, or derivative products, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### ii. Formulations For Enteral Administration

TF and PAR-2 antagonists can be formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673).

Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The polypeptide antagonists may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers, of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perking agents.

Controlled release oral formulations may be desirable. The TF or PAR-2 antagonists can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e. the drug is enclosed in a semipermeable membrane which allows water to enter and push drug out through a single small opening due to osmotic effects. For oral formulations, the location of release may be the stomach, the small intestine (the duodenum, the jejunum, or the ileum), or the large intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcelluose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

### iii. Mucosal Delivery Formulations

Peptide formulations can be administered via the lungs, nasal, oral (sublingual, buccal), vaginal, or rectal mucosa. In cases of pulmonary endometriosis, the TF or PAR-2 antagonists can be delivered to the lungs while inhaling and traverses across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

### iv. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of TF and PAR-2 antagonists, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5, 13-22 (1987); Mathiowitz, et al., Reactive Polymers 6, 275-283 (1987); and Mathiowitz, et al., J. Appl. Polymer Sci. 35, 755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Methods of Manufacture

A. Methods for Producing Polypeptides

Methods for expressing and isolating polypeptides are generally known in the art. Isolated polypeptides can be obtained by, for example, chemical synthesis or by recombinant production in a host cell. To recombinantly produce polypeptides, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a costimulatory polypeptide. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coil strains such as BL-21, and cultured mammalian cells such as CHO cells.

In eukaryotic host cells, a number of viral-based expression systems can be utilized to express polypeptides. Viral based expression systems are well known in the art and include, but are not limited to, baculoviral, SV40, retroviral, or vaccinia based viral vectors.

Mammalian cell lines that stably express variant costimulatory polypeptides can be produced using expression vectors with appropriate control elements and a selectable marker. For example, the eukaryotic expression vectors pCR3.1 (Invitrogen Life Technologies) and p91023(B) (see Wong et al. Science 228:810-815 (1985) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin) The transfected cells can be cultured such that the polypeptide of interest is expressed, and the polypeptide can be recovered from, for example, the cell culture supernatant or from lysed cells. Alternatively, polypeptides can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Polypeptides can be isolated-using, for example, chromatographic methods such as DEAE ion exchange, gel filtration, and hydroxylapatite chromatography. For example, a polypeptide in a cell culture supernatant or a cytoplasmic extract can be isolated using a protein G column. In some embodiments, polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify polypeptides.

B. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse tanscriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis Genetic Engineering News 12:1 (1992); Guatelli et al. Proc. Natl. Acad. Sci. USA 87:1874-1878 (1990); and Weiss, Science 254:1292-1293 (1991).

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

C. Methods for Producing Antibodies

The basic antibody structural unit comprises a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta; or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology, Paul, W., ed., 2nd ed. Raven Press, N.Y., 1989, Ch. 7.

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4.

### i. Production of Polyclonal Antibodies

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography.

### ii. Production Of Monoclonal Antibodies

Monoclonal antibodies may be produced using conventional hybridoma technology, such as the procedures introduced by Kohler and Milstein (Nature, 256:495-97 (1975)), and modifications thereof. An animal, preferably a mouse, is primed by immunization with an immunogen to elicit the desired antibody response in the primed animal. B lymphocytes from the lymph nodes, spleens or peripheral blood of a primed animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfixed parental myeloma cells and donor lymphocyte cells eventually die while only the hybridoma cells survive. These are cloned and grown and their supernatants screened for the presence of antibody of the desired specificity, e.g. by immunoassay techniques. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single monoclonal antibody can be harvested by decantation, filtration, or centrifugation.

a. Production of Chimeric and Humanized Monoclonal Antibodies

Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. In some methods, the isotype of the antibody is human IgG1. IgM antibodies can also be used in some methods. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989), WO 90/07861, U.S. Pat. No. 5,693,762, U.S. Pat. No. 5,693,761, U.S. Pat. No. 5,585,089, U.S. Pat. No. 5,530,101, and Winter, U.S. Pat. No. 5,225,539). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid:

(1) noncovalently binds antigen directly,

(2) is adjacent to a CDR region,

(3) otherwise interacts with a CDR region (e.g. is within about 6 A of a CDR region), or

(4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

b. Production of Human Monoclonal Antibodies

Human antibodies against TF and PAR-2 can be produced by a variety of techniques described below. Some human antibodies are selected by competitive binding experiments or otherwise, to have the same epitope specificity as a particular mouse antibody. Human antibodies preferably have isotype specificity human IgG1.

One method for producing human monoclonal antibodies is the trioma methodology. The basic approach and an exemplary cell fusion partner, SPAZ4, for use in this approach have been described by Oestberg et al., Hybridoma 2:361-367 (1983); Oestberg, U.S. Pat. No. 4,634,664; and Engleman et al., U.S. Pat. No. 4,634,666). The antibody-producing cell lines obtained by this method are called triomas, because they are descended from three cells-two human and one mouse. Initially, a mouse myeloma line is fused with a human B-lymphocyte to obtain a non-antibody-producing xenogeneic hybrid cell, such as the SPAZ4 cell line. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with TF or PAR-2 immunogenic polypeptides. In some methods, B cells are isolated from the same patient who is ultimately to be administered antibody therapy. For in vitro immunization, B-lymphocytes are typically exposed to antigen for a period of 7-14 days in a media such as RPMI-1640 supplemented with 10% human plasma.

The immunized B-lymphocytes are fused to a xenogeneic hybrid cell such as SPAZ-4 by well known methods. For example, the cells are treated with 40-50% polyethylene glycol of MW 1000-4000, at about 37° C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to TF or PAR-2. Triomas producing human antibodies having the desired specificity are subcloned by the limiting dilution technique and grown in vitro in culture medium. The trioma cell lines obtained are then tested for the ability to bind TF or PAR-2.

Although triomas are genetically stable they do not produce antibodies at very high levels. Expression levels can be increased by cloning antibody genes from the trioma into one or more expression vectors, and transforming the vector into standard mammalian, bacterial or yeast cell lines.

Human antibodies against TF and PAR-2 can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/1222, U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741. Transgenic mice are particularly suitable. Anti-TF and anti-PAR-2 antibodies are obtained by immunizing a transgenic nonhuman mammal with polypeptides corresponding to full length TF or PAR-2 polypeptides or immunogenic fragments thereof. Monoclonal antibodies are prepared by, e.g., fusing B-cells from such mammals to suitable myeloma cell lines using conventional Kohler-Milstein technology. Human polyclonal antibodies can also be provided in the form of serum from humans immunized with an immunogenic agent. Optionally, such polyclonal antibodies can be concentrated by affinity purification using TF or PAR-2 polypeptides or fragments thereof as an affinity reagent.

A further approach for obtaining human anti-TF and anti-PAR-2 antibodies is to screen a DNA library from human B cells according to the general protocol outlined by Huse et al., Science, 246:1275-1281 (1989). As described for trioma methodology, such B cells can be obtained from a human immunized with full length TF or PAR-2 polypeptides or immunogenic fragments thereof. Optionally, such B cells are obtained from a patient who is ultimately to receive antibody treatment. Antibodies binding to TF, PAR-2, or fragments thereof are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332). In these methods, libraries of phage are produced in which members display different antibodies on their outer surfaces. Antibodies are usually displayed as Fv or Fab fragments. Phage displaying antibodies with a desired specificity are selected by affinity enrichment to a TF or PAR-2 polypeptide or fragment thereof.

In a variation of the phage-display method, human antibodies having the binding specificity of a selected murine antibody can be produced (Winter, WO 92/20791). In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for αSyn (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for TF or PAR-2 are selected. These phage display the variable regions of completely human anti-TF or anti-PAR-2 antibodies. These antibodies usually have the same or similar epitope specificity as the marine starting material.

The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG1 and IgG3 have complement activity and isotypes IgG2 and IgG4 do not Choice of isotype can also affect passage of antibody into the brain. Human isotype IgG1 is preferred. Light chain constant regions can be lambda or kappa. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

### iii. Expression of Recombinant Antibodies

Chimeric, humanized and human antibodies are typically produced by recombinant expression. Recombinant polynucleotide constructs typically include an expression control sequence operably linked to the coding sequences of antibody chains, including naturally associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof (Winnacker, From Genes to Clones, VCH Publishers, N.Y., 1987). A number of suitable host cell lines capable of secreting intact heterologous proteins have been developed in the art, and include CHO cell lines, various COS cell lines, HeLa cells, L cells, human embryonic kidney cell, and myeloma cell lines. Preferably, the cells are nonhuman. Expression vectors for these cells can include expression control sequences, such as an origin of replication, a promoter, an enhancer (Queen et al., Immunol. Rev., 89:49 (1986)), and necessary processing information sites, such as ribosome binding sites, RNA splice sites, polyadenylation sites, and transcriptional terminator sequences. Preferred expression control sequences are promoters derived from endogenous genes including cytomegalovirus, SV40, adenovirus, bovine papillomavirus (Co et al., J. Immunol., 148:1149 (1992).

Alternatively, antibody coding sequences can be incorporated in transgenes for introduction into the genome of a transgenic animal and subsequent expression in the milk of the transgenic animal (see, e.g., U.S. Pat. No. 5,741,957; U.S. Pat. No. 5,304,489, U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed antibodies can be purified according to standard procedures of the art, including HPLC purification, column chromatography, gel electrophoresis and the like (see generally, Scopes, Protein Purification (Springer-Verlag, N.Y., 1982)).

Polypeptide immunogens disclosed herein can also be linked to a suitable carrier molecule to form a conjugate which helps elicit an immune response. Suitable carriers include serum albumins, keyhole limpet hemocyanin, immunoglobulin molecules, thyroglobulin, ovalbumin, tetanus toxoid, or a toxoid from other pathogenic bacteria, such as diphtheria, E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-S-glycerine cysteine (Pam.sub.3Cys), mannan (a manose polymer), or glucan (a beta 1.fwdarw.2 polymer)), cytokines (e.g., IL-1, IL-1 alpha and beta peptides, IL-2, gamma-INF, IL-10, GM-CSF), and chemokines (e.g., MIP1alpha and beta, and RANTES). Immunogenic agents can also be linked to peptides that enhance transport across tissues, as described in O'Mahony, WO 97/17613 and WO 97/17614. Immunogens may be linked to the carries with or with out spacers amino acids (e.g., gly-gly).

Some conjugates can be formed by linking agents to at least one T cell epitope. Some T cell epitopes are promiscuous while other T cell epitopes are universal. Promiscuous T cell epitopes are capable of enhancing the induction of T cell immunity in a wide variety of subjects displaying various HLA types. In contrast to promiscuous T cell epitopes, universal T cell epitopes are capable of enhancing the induction of T cell immunity in a large percentage, e.g., at least 75%, of subjects displaying various HLA molecules encoded by different HLA-DR alleles.

A large number of naturally occurring T-cell epitopes exist, such as, tetanus toxoid (e.g., the P2 and P30 epitopes), Hepatitis B surface antigen, pertussis, toxoid, measles virus F protein, Chlamydia trachomitis major outer membrane protein, diphtheria toxoid, Plasmodium falciparum circumsporozite T, Plasmodium falciparum CS antigen, Schistosoma mansoni triose phosphate isomersae, Escherichia coli TraT, and Influenza virus hemagluttinin (HA). The immunogenic peptides of the invention can also be conjugated to the T-cell epitopes described in Sinigaglia et al., Nature, 336:778-780 (1988); Chicz R. M. et al., J. Exp. Med., 178:27-47 (1993); Hammer, et al., Cell 74:197-203 (1993); Falk K. et al., Immunogenetics, 39:230-242 (1994); WO 98123635; and, Southwood, et al. J. Immunology, 160:3363-3373 (1998).

Alternatively, the conjugates can be formed by linking agents to at least one artificial T-cell epitope capable of binding a large proportion of MHC Class II molecules., such as the pan DR epitope (“PADRE”). PADRE is described in U.S. Pat. No. 5,736,142, WO 95/07707, and Alexander J et al., Immunity, 1:751-761 (1994). A preferred PADRE peptide is AKXVAAWTLKAAA (SEQ ID NO: 2), wherein X is preferably cyclohexylalanine, tyrosine or phenylalanine, with cyclohexylalanine being most preferred.

Immunogenic agents can be linked to carriers by chemical crosslinking. Techniques for linking an immunogen to a carrier include the formation of disulfide linkages using N-succinimidyl-3-(2-pyridyl-thio)propionate (SPDP) and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. A variety of such disulfide/amide-forming agents are described by Immun. Rev. 62, 185 (1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl)cy-clohexane-1-carboxylic acid. The carboxyl groups can be activated by combining them with succinimide or 1-hydroxyl-2-nitro-4-sulfonic acid, sodium salt.

Immunogenicity can be improved through the addition of spacer residues (e.g., Gly-Gly) between the Tₕ epitope and the peptide immunogen. In addition to physically separating the Tₕ epitope from the B cell epitope (i.e., the peptide immunogen), the glycine residues can disrupt any artificial secondary structures created by the joining of the Tₕ epitope with the peptide immunogen, and thereby eliminate interference between the T and/or B cell responses. The conformational separation between the helper epitope and the antibody eliciting domain thus permits more efficient interactions between the presented immunogen and the appropriate Tₕ and B cells.

To enhance the induction of T cell immunity in a large percentage of subjects displaying various HLA types to an agent of the present invention, a mixture of conjugates with different Tₕ cell epitopes can be prepared. The mixture may contain a mixture of at least two conjugates with different Tₕ cell epitopes, a mixture of at least three conjugates with different Tₕ cell epitopes, or a mixture of at least four conjugates with different Tₕ cell epitopes. The mixture may be administered with an adjuvant.

Immunogenic peptides can also be expressed as fusion proteins with carriers (i.e., heterologous peptides). The immunogenic peptide can be linked at its amino terminus, its carboxyl terminus, or both to a carrier. Optionally, multiple repeats of the immunogenic peptide can be present in the fusion protein. Optionally, an immunogenic peptide can be linked to multiple copies of a heterologous peptide, for example, at both the N and C termini of the peptide. Some carrier peptides serve to induce a helper T-cell response against the carrier peptide. The induced helper T-cells in turn induce a B-cell response against the immunogenic peptide linked to the carrier peptide.

### IV. Methods for Inhibiting or Treating Endometriosis

Although TF is expressed on perivascular cells of normal tissues and in the adventitial layer of blood vessels, these cells are sequestered from contact with circulating fVII by the tight endothelial cell layer of the normal vasculature. Therefore, differential expression of TF by endometriotic tissue makes it a specific target for inhibiting or treating endometriosis. Similarly, overexpression of PAR-2 by endometrial tissue in women with endometriosis makes it a target for inhibiting or treating endometriosis.

In one embodiment, interference with binding of fVII to TF or of the TF/fVIIa to PAR-2 is accomplished by providing one or more antagonists that reduce or inhibit binding of these proteins as described above. In another embodiment, the catalytic activity of PAR-2 or the TF/fVIIa complex is inhibited by providing one or more antagonists as disclosed above. In another embodiment, TF and/or PAR-2 expression is downregulated by providing one or more inhibitory nucleic acids including, but not limited to, ribozymes, triplex-forming oligonucleotides (TFOs), antisense DNA, external guide sequences (EGSs), siRNA, and microRNA specific for nucleic acids encoding TF or PAR-2. TF and PAR-2 antagonists can also be provided in combination with other anti-angiogenic agents or other agents used to treat endometriosis, such as those described above.

In general, the compositions disclosed above are useful for treating a subject having or being predisposed to endometriosis. The terms “treat” and “treating”, as used herein includes alleviating, ameliorating inhibiting and/or eliminating one or more symptoms associated with endometriosis. Symptoms associated with endometriosis include, but are not limited to, dysmenorrhea, chronic pelvic pain, pain on defecation, diarrhea, pain with ovulation, fatigue as well as the sequelae of infertility and depression. In another embodiment, the compositions and methods disclosed herein are useful to reduce the number and/or size of endometriotic lesions in a subject being treated.

The compositions and methods disclosed herein can be used for prophylactic and therapeutic applications. In prophylactic applications, TF and PAR-2 antagonists are administered in amounts and frequencies of administration sufficient to eliminate or reduce the risk or delay the outset of endometriosis, including physiological, biochemical, histologic and/or other symptoms of the disorder, its complications and intermediate pathological phenotypes presenting during development of the disease or disorder. In therapeutic applications, the compositions and methods disclosed herein are administered to a patient suspected of, or already suffering from endometriosis to treat, at least partially, the symptoms of the disease physiological, biochemical, histologic and/or other symptoms), including its complications and intermediate pathological phenotypes in development of the disease or disorder. An amount adequate to accomplish therapeutic or prophylactic treatment is defined as a therapeutically- or prophylactically-effective amount.

The outcome of the therapeutic and prophylactic methods disclosed herein is to at least produce in a patient a healthful benefit, which includes, but is not limited to, prolonging the onset of symptoms of endometriosis, and/or alleviating a symptom of endometriosis after onset of a symptom of the disorder.

TF and PAR-2 antagonists can be used to reduce or inhibit the expression level or activity of TF or PAR-2 on endometrial cells in patients with endometriosis. The TF and PAR-2 antagonists can be any of those described herein, including polypeptides containing any of the disclosed amino acid alterations, polypeptide fragments, fusion proteins and combinations thereof.

As discussed above, endometriosis requires angiogenesis, and tissue factor and PAR-2 participate in angiogenic signaling. In one embodiment, administration of TF and PAR-2 antagonists is effective to inhibit angiogenesis required for the establishment, growth, and persistence of endometriotic lesions.

In a preferred embodiment, the TF or PAR-2 antagonist compositions used to treat endometriosis are Fc fusion proteins. The examples below demonstrate that immunoconjugate fusion proteins containing fVIIa targeting domains fused with immunoglobulin Fc domains are effective to treat pre-established endometriotic lesions in mice, reducing the total number of endometriotic lesions, and their size. While other anti-angiogenic treatments can inhibit the formation of new endometriotic lesions, they have not proven effective at treating existing lesions.

Fc fusion proteins are advantageous for several reasons. First, the Fc domain provides cysteine residues that participate in disulfide bonds and cause the fusion polypeptides to dimerize, thereby increasing the valency of the first fusion partner. The increased valency of the first fusion partner causes an increased avidity of the fusion protein for the cellular target. Additionally, Fc domains recruit molecules and cells of the immune system that bind to Fc, such as macrophages, NK cells, and complement factors that can initiate powerful cytolytic responses to cells that are bound by the Fc fusion protein. Thus, Fc fusion proteins are effective to inhibit the formation of new endometriotic lesions, and are additionally useful to treat pre-existing endometriotic lesions.

A. Methods of Administration of TF and PAR-2 Antagonists

In some in vivo approaches, a TF or PAR-2 antagonist itself is administered to a subject in a therapeutically effective amount. Typically, the polypeptides can be suspended in a pharmaceutically-acceptable carrier. Pharmaceutically acceptable carriers are biologically compatible vehicles (e.g., physiological saline) that are suitable for administration to a human. A therapeutically effective amount is an amount of a TF or PAR-2 antagonist that is capable of producing a medically desirable result (e.g., reduction in symptoms) in a treated animal. TF or PAR-2 antagonists can be administered orally or by intravenous infusion, or injected subcutaneously, intramuscularly, intraperitoneally, intrarectally, intravaginally, intranasally, intragastrically, intratracheally, or intrapulmonarily. The TF and PAR-2 antagonists can be delivered directly to an appropriate tissue or organ (e.g., the endometrium).

In a preferred embodiment, the TF and PAR-2 antagonists are administered intraperitoneally.

B. Methods of Administration of Nucleic Acids

Nucleic acids encoding polypeptide TF or PAR-2 antagonists can be administered to subjects in need thereof. TF or PAR-2 inhibitory nucleic acids as described above can also be administered to subjects in need thereof. Nucleic acid delivery involves introduction of “foreign” nucleic acids into a cell and ultimately, into a live animal. Several general strategies for gene therapy have been studied and have been reviewed extensively (Yang, N-S., Crit. Rev. Biotechnol. 12:335-356 (1992); Anderson, W. F., Science 256:808-813 (1992); Miller, A. S., Nature 357; 455-460 (1992); Crystal, R. G., Amer. J. Med. 92(suppl 6A):44S-52S (1992); Zwiebel, J. A. et al., Ann. N.Y. Acad. Sci. 618:394-404 (1991); McLachlin, J. R. et al., Prog. Nuc. Acid Res. Molec. Biol. 38:91-135 (1990); Kohl, D. B. et al., Cancer Invest. 7:179-192 (1989).

Nucleic acid therapy can be accomplished by direct transfer of a functionally active DNA into mammalian somatic tissue or organ in vivo. For example, nucleic acids encoding TF or PAR-2 antagonists can be administered directly to endometrial tissues. Alternatively, endometrial tissue specific targeting can be achieved using endometrial tissue-specific transcriptional regulatory elements (TREs).

DNA transfer can be achieved using a number of approaches described below. These systems can be tested for successful expression in vitro by use of a selectable marker (e.g., G418 resistance) to select transfected clones expressing the DNA, followed by detection of the presence of the TF or PAR-2 antagonist expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

Examples of successful “gene transfer” reported in the art include: (a) direct injection of plasmid DNA into mouse muscle tissues, which led to expression of marker genes for an indefinite period of time (Wolff, et al., Science, 247:1465 (1990); Acsadi, et al., The New Biologist, 3:71 (1991)); (b) retroviral vectors are effective for in vivo and in situ infection of blood vessel tissues; (c) portal vein injection and direct injection of retrovirus preparations into liver effected gene transfer and expression in vivo (Horzaglou, et al., J. Biol. Chem., 265:17285 (1990); Koleko, et al., Human Gene Therapy, 2:27 (1991); Ferry, et al., Proc. Natl. Acad. Sci. USA, 88:8387 (1991)); (d) intratracheal infusion of recombinant adenovirus into lung tissues was effective for in vivo transfer and prolonged expression of foreign genes in lung respiratory epithelium (Rosenfeld, et al., Science, 252:431 (1991); (e) Herpes simplex virus vectors achieved in vivo gene transfer into brain tissue (Ahmad, F. et al., eds, Miami Short Reports—Advances in Gene Technology: The Molecular Biology of Human Genetic Disease, Vol 1, Boerringer Manneheim Biochemicals, USA, 1991).

Retroviral-mediated human therapy utilizes amphotrophic, replication-deficient retrovirus systems (Temin, H. M., Human Gene Therapy 1:111 (1990); Temin et al., U.S. Pat. No. 4,980,289; Temin et al., U.S. Pat. No. 4,650,764; Temin et al., U.S. Pat. No. 5,124,263; Wills, J. W. U.S. Pat. No. 5,175,099; Miller, A. D., U.S. Pat. No. 4,861,719). Such vectors have been used to introduce functional DNA into human cells or tissues, for example, the adenosine deaminase gene into lymphocytes, the NPT-II gene and the gene for tumor necrosis factor into tumor infiltrating lymphocytes. Retrovirus-mediated gene delivery generally requires target cell proliferation for gene transfer (Miller, D. G. et al., Mol. Cell. Biol. 10:4239 (1990). This condition is met by certain of the preferred target cells into which the present DNA molecules are to be introduced, i.e., actively growing tumor cells. Gene therapy of cystic fibrosis using transfection by plasmids using any of a number of methods and by retroviral vectors has been described by Collins et al., U.S. Pat. No. 5,240,846.

Nucleic acid molecules encoding TF or PAR-2 antagonists may be packaged into retrovirus vectors using packaging cell lines that produce replication-defective retroviruses, as is well-known in the art (see, for example, Cone, R. D. et al., Proc. Natl. Acad. Sci. USA 81:6349-6353 (1984); Mann, R. F. et al., Cell 33:153-159 (1983); Miller, A. D. et al., Molec. Cell. Biol. 5:431-437 (1985); Sorge, J., et al., Molec. Cell. Biol. 4:1730-1737 (1984); Hock, R. A. et al., Nature 320:257 (1986); Miller, A. D. et al., Molec. Cell. Biol. 6:2895-2902 (1986). Newer packaging cell lines which are efficient and safe for gene transfer have also been described (Bank et al., U.S. Pat. No. 5,278,056).

This approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Other virus vectors may also be used, including recombinant adenoviruses (Horowitz, M. S., In: Virology, Fields, B N et al., eds, Raven Press, New York, 1990, p. 1679; Berkner, K. L., Biotechniques 6:616 9191988), Strauss, S. E., In: The Adenoviruses, Ginsberg, H S, ed., Plenum Press, New York, 1984, chapter 11), herpes simplex virus (HSV) for neuron-specific delivery and persistence. Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R. J. et al., EMBO J. 10:3941 (1991).

Another vector which can express the disclosed DNA molecule and is useful in the present therapeutic setting, particularly in humans, is vaccinia virus, which can be rendered non-replicating (U.S. Pat. Nos. 5,225,336; 5,204,243; 5,155,020; 4,769,330; Sutter, G et al., Proc. Natl. Acad. Sci. USA (1992) 89:10847-10851; Fuerst T. R. et al., Proc. Natl. Acad. Sci. USA (1989) 86:2549-2553; Falkner F. G. et al., Nucl. Acids Res (1987) 15:7192; Chakrabarti, S et al., Molec. Cell. Biol. (1985) 5:3403-3409). Descriptions of recombinant vaccinia viruses and other viruses containing heterologous DNA and their uses in immunization and DNA therapy are reviewed in: Moss, B., Curr. Opin. Genet. Dev. (1993) 3:86-90; Moss, B. Biotechnology (1992) 20: 345-362; Moss, B., Curr Top Microbiol Immunol (1992) 158:25-38; Moss, B., Science (1991) 252:1662-1667; Piccini, A et al., Adv. Virus Res. (1988) 34:43-44; Moss, B. et al., Gene Amplif Anal (1983) 3:201-213.

In addition to naked DNA or RNA, or viral vectors, engineered bacteria may be used as vectors. A number of bacterial strains including Salmonella, BCG and Listeria monocytogenes (LM) (Hoiseth & Stocker, Nature 291, 238-239 (1981); Poirier, T P et al. J. Exp. Med. 168, 25-32 (1988); (Sadoff, J. C., et al., Science 240, 336-338 (1988); Stover, C. K., et al., Nature 351, 456-460 (1991); Aldovini, A. et al., Nature 351, 479-482 (1991); Schafer, R, et al., J. Immunol. 149, 53-59 (1992); Ikonomidis, G. et al., J. Exp. Med. 180, 2209-2218 (1994)).

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N.-S., et al., Proc. Natl. Acad, Sci. USA 87:9568 (1990); Williams, R. S. et al., Proc. Natl. Acad. Sci. USA 88:2726 (1991); Zelenin, A. V. et al., FEBS Lett. 280:94 (1991); Zelenin, A. V. et al., FEBS Lett. 244-65 (1989); Johnston, S. A. et al., In Vitro Cell. Dev. Biol. 27:11 (1991)). Furthermore, electroporation, a well-known means to transfer genes into cell in vitro, can be used to transfer DNA molecules to tissues in vivo (Titomirov, A. V. et al., Biochim. Biophys. Acta 1088:131 ((1991)).

“Carrier mediated gene transfer” has also been described (Wu, C. H. et al., J. Biol. Chem. 264:16985 (1989); Wu, G. Y. et al., S. Biol. Chem. 263:14621 (1988); Soriano, P. et al., Proc. Natl. Acad. Sci. USA 80:7128 (1983); Wang, C-Y. et al., Proc. Natl. Acad. Sci. USA 84:7851 (1982); Wilson, J. M. et al., J. Biol. Chem. 267:963 (1992)). Preferred carriers are targeted liposomes (Nicolau, C. et al., Proc. Natl. Acad. Sci. USA 80:1068 (1983); Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA.

Plasmid DNA used for transfection or microinjection may be prepared using methods well-known in the art, for example using the Qiagen procedure (Qiagen), followed by DNA purification using known methods, such as the methods exemplified herein.

C. Dosages

For TF and PAR-2 antagonists and nucleic acids encoding TF and PAR-2 antagonists are appropriate at dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, on the severity and extent of disease, and on the duration of the treatment desired. Generally dosage levels of 0.001 to 10 mg/kg of body weight daily are administered to mammals. Hu Z, Garen Proc Natl Acad Sci USA. 2001; 98:12180-5, reports a dosage range of 5 micrograms protein which was injected systemically into mice (average weight of most laboratory mice is 20 g) for treatment of melanoma. Generally, the dosage for intraperitoneal or intravenous injection or infusion will be lower. However, the mouse data provides a starting point for the dosage based on a weight basis.

### IV. Methods for Detecting or Diagnosing Endometriosis

The discovery that tissue factor and PAR-2 are overexpressed in endometrial tissues from women with endometriosis provides new markers for diagnosing endometriosis and/or determining the clinical stage of endometriosis in a subject, or response to therapeutic treatment.

To detect or diagnose endometriosis in a subject, baseline values for the expression levels of TF and PAR-2 are established in order to provide a basis for the diagnosis and/or clinical staging of endometriosis in a subject. In some embodiments, this is accomplished by determining the level of expression of TF or PAR-2 in a sample of bodily fluids, tissue biopsies, or cell extracts taken from normal subjects (endometriosis-free subjects). In one embodiment, the sample to be tested is peritoneal fluid. In another embodiment, the sample to be tested includes peritoneal macrophages and/or monocytes. In another embodiment, the sample to be tested is a biopsy of endometrium.

The samples are reacted with one or more reagents including polypeptides, antibodies and nucleic acids that bind to TF or PAR-2 polypeptides or nucleic acids encoding the same under conditions suitable for complex formation. Such conditions are well known in the art. Differential expression levels of TF or PAR-2 in the test sample as compared to the control samples can be used to diagnose endometriosis or determine the stage or severity of the condition. Determining the level of expression of TF or PAR-2 in a patient can also be useful to determine therapeutic approaches to be used to treat endometriosis in a subject.

As will be appreciated by those in the art, evaluation of expression levels of TF and PAR-2 may be done by evaluation at either the gene transcript, or the protein level; that is, the amount of gene expression may be monitored using nucleic acid probes to the RNA of the gene transcript, and the quantification of gene expression levels, or, alternatively, the final gene product itself (protein) can be monitored, for example through the use of antibodies to the protein and standard immunoassays (ELISAs, etc.) or other techniques, including mass spectroscopy assays, 2D gel electrophoresis assays, etc. Techniques for determining expression levels of RNA include, but are not limited to, quantitative reverse transcriptase PCR, Northern analysis and RNase protection.

In some embodiments, antibodies, polypeptides or nucleic acids specific for TF or PAR-2 can be used for in situ imaging techniques. In this method, cells are contacted with the nucleic acid or polypeptide reagent. Following washing to remove non-specific binding, the presence of the nucleic acid or polypeptide is detected. In one embodiment the nucleic acid or polypeptide contains a detectable label. In another embodiment, the nucleic acid or polypeptide bound to the sample is detected by adding a secondary reagent that contains a detectable label and binds to the primary reagent. This is customarily done when antibodies are used as the primary reagent.

In some embodiments the label is detected in a fluorometer which has the ability to detect and distinguish emissions of different wavelengths. In addition, a fluorescence activated cell sorter (FACS) can be used in the method.

In some embodiments, expression levels of TF or PAR-2 can be used to determine the stage or severity of endometriosis in a patient.

The efficacy of therapeutic agents can also be determined using the diagnostic assays described above. As will be appreciated by a person of skill in the art, assays to determine the efficacy of a therapeutic agent require the establishment of baseline values. In some embodiments, this is accomplished by determining the level of expression of TF or PAR-2 in samples from a patient with endometriosis prior to treatment. Levels of TF and PAR-2 expression after treatment can then be compared to expression levels prior to treatment to determine the efficacy of the treatment.

### EXAMPLES

### Example 1

### Differential Expression of Tissue Factor in Normal and Endometriotic Lesions

Materials and Methods:

Peroxidase staining was performed on 5 micron sections of paraffin-embedded tissues. Staining for TF was conducted with anti-TF (R&D) at a dilution of 1:100. Negative control slides were pre-absorbed with 50-molar excess of the corresponding peptides or pre-immune serum for 2 hr. at room temperature. Treatment with the appropriate peroxidase conjugate and color development with DAB was be carried out using the Vectastain ABC kit (Vector Laboratories, Burlingame, Calif.). Samples were counterstained with hematoxylin. Visualization and photography was conducted with an inverted contrast microscope (Olympus, Mellville, N.Y.),

Results:

Prior immunohistochemistry (IHC) and in situ hybridization studies as well as in vitro experiments demonstrate that in normal endometrium, progesterone (P4) markedly enhances tissue factor expression in decidualized stromal cells during the secretory phase while glands display minimal tissue factor expression throughout the menstrual cycle (Lockwood, et al., J. Clin. Endocrinol. Metab., 76(1):231-6 (1993); Krikun, et al., J. Clin. Endocrinol. Metab., 83(3):926-30 (1998); Lockwood, et al., J. Clin. Endocrinol. Metab., 85(1):297-301 (2000); Krikun, et al., Mol. Endocrinol., 14(3):393-400 (2000); Runic, et al., J. Clin. Endocrinol. Metab., 82(6):1983-8 (1997)). By contrast, it is now demonstrated that tissue factor expression has a strikingly different pattern in eutopic and ectopic endometrium derived from women with endometriosis. In these tissues tissue factor expression is greatly elevated in the glands throughout the menstrual cycle.

The IHC data demonstrate tissue factor immunostaining in decidualized stromal cells, but not in glandular epithelium of normal secretory endometrium. In contrast, the data demonstrate over-expression of tissue factor in glands and stromal cells in eutopic late proliferative phase endometrium from patients with endometriosis. The data also demonstrate intense tissue factor staining in glandular epithelium and stromal cells in ectopic endometriotic implants from proliferative phase endometrium.

### Example 2

### Differential Expression of PAR-2 in Normal and Endometriotic Lesions

Materials and Methods:

Peroxidase staining was performed on 5 micron sections of paraffin-embedded tissues. Staining for PAR2 was conducted with anti-PAR 2 (Santa Cruz Biotechnology, Santa Cruz, Calif.) at a dilution of 1:100. Negative control slides were pre-absorbed with a 50-molar excess of the corresponding peptides or pre-immune serum for 2 hr. at room temperature. Treatment with the appropriate peroxidase conjugate and color development with DAB was carried out using the Vectastain ABC kit (Vector Laboratories, Blurlingame, Ca). Samples were counterstained with hematoxylin. Visualization and photography was conducted with an inverted contrast microscope (Olympus, Mellville, N.Y.).

Results:

Normal secretory endometrium and eutopic and ectopic endometrium from patients with endometriosis was also tested for expression levels of PAR-2 by immunohistochemistry. The data demonstrate minimal glandular and stromal PAR-2 immunostaining in normal secretory endometrium. In contrast, the data demonstrate greatly upregulated expression of PAR-2 in glandular epithelial and endothelial cells of eutopic and ectopic endometrium from patients with endometriosis compared with normal eutopic controls. Expression of PAR-2 in stromal cells was variable, displaying a range of immunostaining.

### Example 3

### Expression of Tissue Factor and PAR-2 in Macrophages from Endometriotic Lesions

Materials and Methods:

The materials and methods used were generally as described above with respect to Example 1 and Example 2. The anti-CD68 was obtained from (Dako, Carpinteria, Calif.). This antibody detects macrophages and is specific for immunohistochemistry of frozen or paraffin fixed sections.

Results:

Immunohistochemistry of endometriotic tissue was also performed using an antibody that recognizes the macrophage specific marker CD68. Comparison of serial sections stained with anti-tissue factor, anti-PAR-2 and anti-CD68 antibodies revealed that tissue factor and PAR-2 are highly expressed in infiltrating macrophages in ectopic endometriotic tissues.

### Example 4

### Effects of Treatment with Tissue Factor-Specific Fc Fusion Protein on Endometriotic Lesions in Mice

Materials and Methods:

Synthesis of Icon protein was carried out in transfected CHO cells. The procedure for transfecting the pcDNA3.1(+) plasmid vectors for the Icon into CHO cells and isolating stably transfected clones is described by Hu, Sun & Garen Proc Natl Acad Sci USA. 1999 Jul. 6; 96(14):8161-6; Hu & Garen Proc Natl Acad Sci USA. 2000 Aug. 1; 97(16):9221-5, Proc Natl Acad Sci USA. 2001 Oct. 9; 98(21):12180-5) with modifications as follows. The transfected CHO cells were cultured in serum-free CHO medium (EXCELL 301, JRH Biosciences) supplemented with a final concentration of 1 μg/ml of vitamin K1 (Sigma). The Icon protein was purified from the culture medium by affinity chromatography on HiTrap rProtein A FF 5 ml column (Amersham Biosciences), dialyzed against 10 mM HEPES pH7.4, 150 mM NaCl2, and 5 mM CaCl2, and then concentrated by centrifugal device (MW CO 100,000, Millipore). The final concentration of the Icon was determined with the Bradford assay reagent (Bio-Rad).

Significance was determined with the Kruskal-Wallis One Way Analysis of Variance on Ranks. All pair-wise multiple comparison procedures were conducted by Dunn's Method. P values denote the appropriate significance.

Results:

Athymic, ovariectomized and estradiol-treated mice received intraperitoneal (i.p.) injections of 1.0 mg of proliferative phase human eutopic endometrial tissue. Tissues were derived from women undergoing hysterectomy for benign conditions not affecting the endometrium. Twelve days after inoculation, to permit nidation and to establish lesions, Icon protein (5 or 10 μg) was delivered i.p. once a week for 4 weeks. After sacrifice, animals were subjected to gross inspection. The results are presented in Table 1, which indicates that administration of the Icon fusion protein produced a dose-dependent reduction in both size and numbers of lesions, with the high concentration of Icon (10 μg) abolishing the presence of all lesions in 11 of 15 mice (Table 1) and reducing both size and number of lesions in the remaining mice.

TABLE 1
Effect of 5 or 10 μg of Icon fusion protein in the reduction of
endometriotic implants.

| | #mice | | | Avg | | | |
|---|---|---|---|---|---|---|---|
| Mouse | w/disease/ | % w/ | p | size/ | p | Avg | p |
| Treatment | total number | disease | value | total | value | #/total | value |
| E2 (CTL) | 12/13 | 92% | | 2.88 | | 1.6 | |
| E2 + Icon | 7/12 | 58% | NS | 1.21 | <0.05 | 1.6 | NS |
| (5 ug) | | | | | | | |
| E2 + Icon | 4/15 | 27% | <0.05 | 1.5 | <0.05 | 1.0 | <0.05 |
| (10 ug) | | | | | | | |

E2 = Estradiol,
NS = not statistically significant.

Residual endometriotic lesions were formalin fixed, paraffin-embedded and immunostained for von Willebrand's factor (vWF) with polyclonal antibody Ab6994 from Abeam (Cambridge, Mass.) which recognizes both human and mouse antigens. Immunostaining was evaluated by two independent observers employing a semi-quantitative method in accordance with the following scoring system: 0, absence of staining; 0/+, presence of weak focal staining; +, moderate staining; ++, marked staining.

The blood vessels in mice with residual lesions after treatment with Icon were smaller, atrophic and displayed significant changes in vessel areas with reductions of 87%+/−26% (p0.002, mean+/−SEM, n=3) compared with controls. Assessment was conducted by 2 independent observers. Moreover, aberrant endometrial morphology was clearly seen in all mice treated with the Icon fusion protein but not in controls including hemosideroin deposition and disrupted glandular architecture. No systemic hemorrhagic or thrombotic sequelae were observed in Icon treated mice.

The results suggest that Fc fusion proteins targeting tissue factor on endometriotic endothelial cells can target both developing and established human endometriotic lesions in athymic mice. The gross and microscopic vessel analysis suggests that these fission proteins directly or indirectly destroy endometriotic vessels. Moreover, there is a clear change in the morphology of the endometrial implant where both the structure of the glands and the stroma are clearly disrupted.

Modifications and variations of the present invention will be obvious to those skilled in the art from the foregoing detailed description and are intended to come within the scope of the appended claims.

1. A method for treating or inhibiting endometriosis in an individual comprising administering a TF or PAR-2 antagonist in an amount effective to reduce or inhibit one or more symptoms associated with endometriosis in the individual.

2. The method of claim 1 wherein the TF or PAR-2 antagonist is a polypeptide.

3. The method of claim 2 wherein the polypeptide is an antibody or a fragment of an antibody that binds to TF or PAR-2.

4. The method of claim 3 wherein the antibody is a monoclonal antibody.

5. The method of claim 2 wherein the TF antagonist is a catalytically inactive fVIIa polypeptide or a fragment thereof that binds to tissue factor.

6. The method of claim 5 wherein the catalytically inactive fVIIa polypeptide comprises one or more covalent active site inhibitors.

7. The method of claim 5 wherein the catalyically inactive fVIIa polypeptide comprises one or more amino acid alterations selected from the group consisting of substitutions, insertions and deletions relative to native human fVIIa.

8. The method of claim 7 wherein the catalytically inactive fVIIa polypeptide comprises one or more amino acid substitutions at amino acid positions 193, 242, 341 and 344 of the native human fVIIa amino acid sequence.

9. The method of claim 8 wherein the catalytically inactive fVIIa polypeptide comprises a K341A substitution.

10. The method of claim 2 wherein the polypeptide comprises a fusion polypeptide comprising:
a) as a first fusion partner, the polypeptide of any of claims 5-9, and
b) as a second fusion partner, a second polypeptide,
wherein the first fusion partner is fused directly to the second fusion partner, or optionally, is fused to a linker sequence that is fused to the second fusion partner.

11. The method of claim 10 wherein the first fusion partner is fused directly to the second fusion partner.

12. The method of claim 10 wherein the second polypeptide comprises one or more domains of an Ig heavy chain constant region.

13. The method of claim 12 wherein the second polypeptide comprises an amino acid sequence corresponding to the hinge, C_{H}2 and C_{H}3 regions of a human immunoglobulin C1 chain.

14. The method of claim 13 wherein the first polypeptide comprises a catalytically inactive fVIIa polypeptide comprising a K341A substitution, and
wherein the second polypeptide comprises an amino acid sequence corresponding to the hinge, CH2 and CH3 regions of a human immunoglobulin C1 chain.

15. The method of claim 1 wherein the TF or PAR-2 antagonist is an inhibitory nucleic acid.

16. The method of claim 15 wherein the inhibitory nucleic acid is selected from the group consisting of ribozymes, triplex-forming oligonucleotides (TFOs), external guide sequences (EGSs) that promote cleavage by RNase P, peptide nucleic acids, antisense DNA, siRNA, and microRNA specific for nucleic acids encoding TF or PAR-2.

17. The method of claim 1 wherein the antagonist is administered intraperitoneally.

18. The method of claim 1 wherein the antagonist is administered to a mucosal surface.

19. The method of claim 1 wherein the antagonist is administered to an individual with endometriosis that has expanded into the peritoneal cavity.

20. The method of claim 1 further comprising treating the individual with one or more additional treatments for endometriosis, selected from the group consisting of oral contraceptive pills, progestin s, GnRH agonists, synthetic androgens, and aromatase inhibitors.

21. The method of claim 1 wherein the individual has been determined to overexpress TF on endometriotic endothelium or endometriotic macrophages, epithelial cells or stromal cells or to overexpress PAR-2 on endometrial tissue.

22. A dosage unit for intraperitoneal or mucosal administration to an individual with endometriosis of a tissue factor or PAR-2 antagonist in an amount effective to reduce or inhibit one or more symptoms associated with endometriosis and a pharmaceutically acceptable carrier.

23. The dosage unit of claim 22 wherein the tissue factor or PAR-2 antagonist is a polypeptide.

24. The dosage unit of claim 22 wherein the polypeptide is an antibody or a fragment of an antibody that binds to TF or PAR-2.

25. The dosage unit of claim 22 wherein the antibody is a monoclonal antibody.

26. The dosage unit of claim 22 wherein the TF antagonist is a catalytically inactive fVIIa polypeptide or a fragment thereof that binds to TF.

27. The dosage unit of claim 26 wherein the catalytically inactive fVIIa polypeptide comprises one or more covalent active site inhibitors.

28. The dosage unit of claim 26 wherein the catalytically inactive fVIIa polypeptide comprises one or more amino acid alterations selected from the group consisting of substitutions, insertions and deletions relative to native human fVIIa.

29. The dosage unit of claim 26 wherein the catalytically inactive fVIIa polypeptide comprises one or more amino acid substitutions at amino acid positions 193, 242, 341 and 344 of the native human fVIIa amino acid sequence.

30. The dosage unit of claim 29 wherein the catalytically inactive fVIIa polypeptide comprises a K341A substitution.

31. The dosage unit of claim 22 wherein the polypeptide comprises a fusion polypeptide comprising:
a) as a first fusion partner, the polypeptide of any of claims 26-30, and
b) as a second fusion partner, a second polypeptide,
wherein the first fusion partner is fused directly to the second fusion partner, or optionally, is fused to a linker sequence that is fused to the second fusion partner.

32. The dosage unit of claim 31 wherein the first fusion partner is fused directly to the second fusion partner.

33. The dosage unit of claim 31 wherein the second polypeptide comprises one or more domains of an Ig heavy chain constant region.

34. The dosage unit of claim 33 wherein the second polypeptide comprises an amino acid sequence corresponding to the hinge, C_{H}2 and C_{H}3 regions of a human immunoglobulin C1 chain.

35. The dosage unit of claim 34 wherein the first polypeptide comprises a catalytically inactive fVIIa polypeptide comprising a K341A substitution, and wherein the second polypeptide comprises an amino acid sequence corresponding to the hinge, CH2 and CH3 regions of a human immunoglobulin C1 chain.

36. A method for diagnosing or assisting in the diagnosis of endometriosis comprising
detecting the level of expression of TF or PAR-2 in a biological sample obtained from an individual to be tested and comparing the level of expression with the level of expression in one or more biological samples obtained from individuals not having endometriosis, wherein the overexpression of TF and/or PAR-2 in the sample from the individual to be tested relative to the samples obtained from individuals not having endometriosis is indicative of endometriosis.
